(19) United States
(12) Patent Application Publication
(10) Pub. No.: US 2025/0002839 A1
(43) Pub. Date: Jan. 2, 2025

(54) Methods, Compositions And Devices For Developing Biophotonic Charge Storage Cells

(71) Applicant: The University Of North Carolina At Greensboro, Greensboro, NC (US)

(72) Inventors: Hemali P. Rathnayake, Summerfield, NC (US); Selina Laws, Durham, NC (US); Tetyana Ignatova, Whitsett, NC (US)

(21) Appl. No.: 18/709,650

(22) PCT Filed: Nov. 18, 2022
(86) PCT No.: PCT/US22/50450

§ 371 Date: May 13, 2024

Related U.S. Application Data
(60) Provisional application No. 63/281,414, filed on Nov. 19, 2021.

(51) Int. Cl. C12N 1/20; H01M 8/16

(52) U.S. Cl. CPC C12N 1/20; H01M 8/16

(57) ABSTRACT

Disclosed are methods, compositions and devices for developing biophotonic charge storage cells. In certain embodiments, the methods, compositions and devices employ interfacial programming of artificial siderophores to create photosynthetic and photoresponsive bacteria and bacteria anchored biophotonic architectures. The biophotonic charge storage cells may be used to power external devices such as a glucose meter.

### FIELD

The presently disclosed subject matter relates to methods, compositions and devices for developing biophotonic charge storage cells.

### BACKGROUND

One of the most significant challenges with sustainable energy systems is how to store electricity that is generated from wind, solar and/or waves. At present, few existing technologies provide for harvesting, conversion, and storage in a single system for sustainable energy at a low financial and environmental cost. One exception may be the use of photosynthetic and phototropic microbes. The use of these microbes or pigment-protein complexes isolated from natural bacterial photosystems that absorb energy across the solar spectrum and transiently store harvested energy through charge separation are in some cases able to store photo-generated charges for a prolong time. For example, photosynthetic purple bacteria may have the ability to provide highly quantum-efficient biophotonic processes with significant impact on energy technologies. However, to date, self-charging biophotonic power cells are not yet capable of producing sufficient photovoltage and capacitance to use as large-scale prolonged charge storage systems. To date known photosystems made from isolated bacterial protein complexes can generate photovoltage of about 0.45 V and capacitance of 0.1 F/m². However, this is significantly below the photovoltage and charge capacity necessary for powering miniature electronic devices other than a light-emitting diode (LED).

Thus, there is a need to develop technologies that provide harvesting, conversion, and storage in a single system for sustainable energy at a low financial and environmental cost and that are capable of generating photovoltage and charge capacity necessary for powering electronic devices other than a LED.

### SUMMARY

Disclosed are methods, compositions and devices for developing biophotonic charge storage cells. In certain embodiments, the methods, compositions and devices employ interfacial programming of artificial siderophores to create photosynthetic and photoresponsive bacteria and bacteria-anchored biophotonic architectures. The methods, devices and systems may be embodied in a variety of ways.

In some embodiments, disclosed are methods of making a photosynthetic power cell. In some embodiments, the methods comprise combining: an amphiphilic siderophore; a metal ion; and a photosynthetic and photoresponsive microorganism, wherein the photosynthetic and photoresponsive microorganism is anchored to the amphiphilic siderophore. In certain embodiments, the method of making a photosynthetic power cell comprises the steps of: (a) forming a layer of artificial metallo-siderophores by combining: (i) an amphiphilic siderophore; (ii) a metal ion; and (iii) a photosynthetic and/or photoresponsive microorganism, wherein the combining is performed such that the microorganism is anchored to the amphiphilic siderophore; and (b) adding the layer of artificial metallo-siderophores to a surface of a power cell

In other embodiments, disclosed are compositions for biophotonic charge storage comprising: an amphiphilic siderophore; a metal ion; and a photosynthetic and/or photoresponsive microorganism, wherein the microorganism is anchored to the amphiphilic siderophore. In certain embodiments, the compositions are made by an embodiment of the disclosed methods.

In yet other embodiments, disclosed are devices for biophotonic storage. In some embodiments, the device may comprise at least a single layer of a siderophore-metal ion coordinated complex with a photosynthetic and/or photoresponsive microorganism, wherein the at least a single siderophore layer is placed between an anode and a cathode.

### BRIEF DESCRIPTION OF THE DRAWINGS

The present disclosure may be better understood by reference to the following non-limiting figures.

FIG. 1 shows chemical structures, and three-dimensional (3D) geometry optimized catecholate type artificial siderophores in accordance with an embodiment of the disclosure.

FIG. 2 shows a scheme depicting the formation of a catecholate Syn-Enterobactin A-Fe(III) complex (left), as well as a crystal unit cell (middle) and space-filling packing pattern (right) of the siderophore-Fe(III) complex in accordance with an embodiment of the disclosure.

FIG. 3 shows chemical structures, and three-dimensional (3D) geometry for optimized hydroxamate type artificial siderophores and a synthetic scheme for their preparation in accordance with an embodiment of the disclosure.

FIG. 4 shows a scheme depicting the formation of hydroxamate Syn-Ferrichorme A-Fe(III) complex (top), and crystal unit cell (bottom left) and space-filling packing pattern (bottom right) of the siderophore-Fe(III) complex in accordance with an embodiment of the disclosure.

FIG. 5 shows structures of dimeric artificial siderophore-metal ion complexes in accordance with an embodiment of the disclosure.

FIG. 6 shows steps of directed self-assembly fabrication process at the liquid-liquid interface (LLI) in accordance with an embodiment of the disclosure.

FIG. 7 illustrates a process for fabricating a miniature prototype with single-layer power cell structure in accordance with an embodiment of the disclosure.

FIG. 8 shows a schematic representation of a device for powering a blood glucose meter in accordance with an embodiment of the disclosure.

FIG. 9a shows a cartoon of a single layer photovoltaic device structure in accordance with an embodiment of the disclosure; FIG. 9b shows a graph of the I-V curve of a test device made from a metallo-pigment loaded artificial siderophore; FIG. 9c is a FITC Epifluorescence microscopy image of S. elongatus bacteria cells; FIG. 9d is a SEM image of S. elongatus cells cultured on an artificial siderophore (no directed self-assembly processes applied); and FIG. 9e is a graph of the I-V curve of a bio-photovoltaic device made from S. elongatus-artificial siderophore bio-hybrid substrate.

FIG. 10a shows the chemical structure of a methyl melamine-silane siderophore and FIG. 10b shows a metal-coordinated methyl melamine-siderophore.

### DETAILED DESCRIPTION

The disclosed subject matter now will be described more fully hereinafter with reference to the accompanying description and drawings, in which some, but not all embodiments of the disclosed subject matter are shown. The disclosed subject matter can be embodied in many different forms and should not be construed as limited to the embodiments set forth herein. Like numbers refer to like elements throughout.

Many modifications and other embodiments of the disclosed subject matter set forth herein will come to mind to one skilled in the art to which the disclosed subject matter pertains having the benefit of the teachings presented in the descriptions and the associated drawings. Therefore, it is to be understood that the presently disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Definitions and Descriptions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. Additionally, any reference referred to as being “incorporated herein” is to be understood as being incorporated in its entirety.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

As used herein, an “amphiphilic siderophore” contains a hydrophilic head group (e.g., a peptide headgroup) and a hydrophobic tail (e.g., a hydrocarbon tail).

As used herein, “anchored” refers to attachment of a siderophore to a microbe. In certain embodiments, such a attachment occurs via selective recognition of proteins, e.g., receptor proteins, on the cell membrane of microbes.

As used herein, the term “biophotonic process” is the interaction of biological matter with photons via photophysical and photochemical processes taking place during the initial steps of photosynthesis.

As used herein, a “biophotovoltaic (BPV) cell” is a cell capable of releasing electrons in the light during the process of oxygenic photosynthesis and in the dark during the oxidation of carbohydrate or other carbon-containing compounds synthesized from carbon dioxide without the need of an added carbon feedstock. In some cases, the BPV device can repair light-induced damage to the photosynthetic apparatus adding to its durability.

As used herein, “catecholate-type siderophore” or “catecholate-type artificial siderophore” refers to molecules that may chelate ferric iron via hydroxyl groups. Siderophores discussed herein may chelate other metal ions.

As used herein, “hydroxamate-type siderophore” or “hydroxamate-type artificial siderophore” refers to molecules that may chelate ferric iron via a carbonyl group in amides. Siderophores discussed herein may chelate other metal ions.

As used herein, “directed self-assembly” refers to the process of self-assembling amphiphilic siderophore-metal ion coordinated complexes at a liquid-liquid interface (LLI) by optimizing conditions to utilize the hydrophobic and hydrophilic interactions of the functional groups, π-π interactions and/or, in some cases, hydrogen bonding, to drive the formation of superstructures of amphiphilic siderophore-metal ion complexes. Conditions may be optimized by using two immiscible solvents (e.g., oil-in water system) where one solvent serves as a good solvent for the complex and one solvent serves as a poor solvent for the complex. The solvent polarity, dielectric nature, acidity and basicity, and possible interfacial interactions with the siderophore's functional groups are relevant factors in solvent selection.

As used herein, “in-situ coordination programming” refers to a process of self-assembling amphiphilic siderophore-metal ion coordinated complexes at a liquid-liquid interface (LLI). In certain embodiments this may be done by first dissolving the amphiphilic siderophore in a polar protic solvent, and catalyzing hydrogen bonding interactions and solvent-solute interactions. Thereafter, a water-oil interface is created initializing metal ion coordination interactions with the amphiphilic siderophores, followed by aggregation of the amphiphilic siderophore-metal ion complexes with the addition of a third non-polar solvent.

As used herein, “siderophores” are low-molecular weight high-affinity metal ion chelators. In nature, siderophores are low-molecular weight iron chelators produced by bacteria and fungi for the uptake of iron as a nutrient, where low-molecular weight can refer to a 2 kD or 2000 (mol/g) cut-off Siderophores discussed herein are not limited to iron interactions.

### Biophotonic Storage Devices

Disclosed are methods, compositions and devices for developing biophotonic charge storage cells. In certain embodiments, the methods, compositions and devices employ interfacial programming of artificial siderophores. In some embodiments, the artificial siderophores create photosynthetic and photoresponsive bacteria and bacteria anchored biophotonic architectures. The methods, compositions and devices may be embodied in a variety of ways.

In certain embodiments, provided are bio-photonic architectures that can be generated by selective anchoring of photosynthetic and photoresponsive microorganisms using self-assembled superstructures of new hydroxamate and catecholate type artificial siderophores. For example, in certain embodiments disclosed are a series of hydroxamate (type I) and catecholate (type II) type artificial siderophores as well as mechanisms of their assembly-disassembly by coordinating with metal ions. Such metal ions may include, but are not limited to at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), and Lawrencium (Lr). In certain embodiments, the metal ion is at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)).

In some embodiments disclosed are structures, metal ion-coordination complexes, and crystal structures of hydroxamate and catecholate type artificial siderophores. Also disclosed are liquid-liquid interface self-assembly processes to create superstructure systems of artificial siderophores. For example, disclosed are directed coordination programming driven self-assembly approaches. In some embodiments, the coordination programming driven self-assembly can by facilitated by a hydrophilic solvent. In some embodiments, the coordination programming driven self-assembly can be facilitated by a hydrophobic solvent. In some embodiments, the coordination programming driven self-assembly can be facilitated by a polar solvent. In some embodiments, the coordination programming driven self-assembly can be facilitated by an apolar solvent.

In some embodiments, the metal-ion-coordination complexes have an octahedral geometry. In some embodiments, the metal-ion-coordination complexes have a tetrahedral geometry. In some embodiments, the metal-ion-coordination complexes have a square planar geometry. In some embodiments, the metal-ion-coordination complexes have an equal combination of octahedral, tetrahedral, or square planar geometries. In some embodiments, the metal-ion-coordination complexes have a non-equal combination of octahedral, tetrahedral, or square planar geometries. In some embodiments, the metal-ion-coordination complex may have a liner, trigonal planar, tetrahedral, square planar, trigonal bipyramidal, square pyramidal, octahedral, trigonic prismatic, pentagonal bipyramidal, capped octahedral, capped trigonal prismatic, square antiprismatic, bisdisphenoid or dodecahedral, bicapped trigonal prismatic, cubic, hexagonal bipyramidal, octahedral trans-bicapped, trigonal prismatic/triangular face bicapped, tricapped trigonal prismatic, capped square antiprismatic, bicapped square antiprismatic, icosahedron, cuboctahedron, anticuboctahedron or triangular orthobicupola, or bicapped hexagonal antiprismatic geometry.

In some embodiments, at least one type of metal ion is introduced to form the metal-ion-coordination complex. In some embodiments, at least two types of metal ions are introduced to form the metal-ion-coordination complex. In some embodiments, at least three types of metal ions are introduced to form the metal-ion-coordination complex. In some embodiments, at least four types of metal ions are introduced to form the metal-ion-coordination complex.

In yet other embodiments, disclosed are artificial siderophores superstructure substrates that may have varying dynamic and adaptive morphologies with respect to different external stimuli (light, temperature, pH, solvent type, metal ion). Further disclosed are bio-photonic architectures-anchored with photosynthetic and photoresponsive bacteria, including Rhodobacter sphaeroides and a cyanobacteria—Synechocystis sp. FCC 6803 (a model organism for bio-photovoltaic systems and bioethanol production). Also disclosed, are device structures of bio-photonic cells such as bio-photovoltaic devices, bio-energy storage devices, bio-hydrogen fuel cells, and bio-photonic sensors. Other embodiments of the disclosure are provided herein.

### Methods of Making a Photosynthetic Power Cell

Embodiments of the present disclosure relate to methods for the development of bio-photonic architectures. In certain embodiments, the method comprises selective anchoring of photosynthetic and photoresponsive microorganisms using self-assembled superstructures of hydroxamate-type and/or catecholate-type artificial siderophores. In certain embodiments, the hydroxamate and/or catecholate type artificial siderophores bind selectively to receptor proteins on the cell membrane of microbes. In certain embodiments, the method provides a plurality of different (e.g., a series) hydroxamate (type I) and catecholate (type II) type artificial siderophores. In yet other embodiments, the disclosed methods employ defined mechanisms for the assembly-disassembly of such siderophores. For example, the siderophores may by coordinated with metal ions, as, for example, transition metals ions such as iron (Fe), zinc (Zn), copper (Cu), and nickel (Ni) in the form of chloride or acetate or other analogous salts thereof. The disclosed siderophores may be embodied in a variety of ways as well as a variety of combinations of new siderophores formed with different metal ions or different combinations of metal ions.

In some embodiments, the method provides self-assembly of hydroxamate-type siderophores in the presence of metal ions. In some embodiments, the method provides self-assembly of catecholate-type siderophores in the presence of metals ions. Or, other types of siderophores may be used. In some embodiments, the method provides a combination of hydroxamate-type and catecholate-type siderophores in the presence of metal ions. In some embodiments the combination ratio of hydroxamate-type and catecholate-type siderophores is 1:1. In some embodiments the combination ratio of hydroxamate-type and catecholate-type siderophores is 1:2. In some embodiments the combination ratio of hydroxamate-type and catecholate-type siderophores is 1:3. In some embodiments the combination ratio of hydroxamate-type and catecholate-type siderophores is 1:4. In some embodiments the combination ratio of hydroxamate-type and catecholate-type siderophores is 1:5. In some embodiments the combination ratio of hydroxamate-type and catecholate-type siderophores is 1:6. In some embodiments the combination ratio of hydroxamate-type and catecholate-type siderophores is 1:7. In some embodiments the combination ratio of hydroxamate-type and catecholate-type siderophores is 1:8. In some embodiments the combination ratio of hydroxamate-type and catecholate-type siderophores is 1:9. In some embodiments the combination ratio of hydroxamate-type and catecholate-type siderophores is 1:10. In some embodiments the combination ratio of hydroxamate-type and catecholate-type siderophores is 1:11. In some embodiments the combination ratio of hydroxamate-type and catecholate-type siderophores is 1:12. In some embodiments the combination ratio of hydroxamate-type and catecholate-type siderophores is 1:13. In some embodiments the combination ratio of hydroxamate-type and catecholate-type siderophores is 1:14. In some embodiments the combination ratio of hydroxamate-type and catecholate-type siderophores is 1:15. In some embodiments the combination ratio of hydroxamate-type and catecholate-type siderophores is 1:16. In some embodiments the combination ratio of hydroxamate-type and catecholate-type siderophores is 1:17. In some embodiments the combination ratio of hydroxamate-type and catecholate-type siderophores is 1:18. In some embodiments the combination ratio of hydroxamate-type and catecholate-type siderophores is 1:19. In some embodiments the combination ratio of hydroxamate-type and catecholate-type siderophores is 1:20.

In some embodiments the combination ratio of hydroxamate-type and catecholate-type siderophores is 2:1. In some embodiments the combination ratio of hydroxamate-type and catecholate-type siderophores is 3:1. In some embodiments the combination ratio of hydroxamate-type and catecholate-type siderophores is 4:1. In some embodiments the combination ratio of hydroxamate-type and catecholate-type siderophores is 5:1. In some embodiments the combination ratio of hydroxamate-type and catecholate-type siderophores is 6:1. In some embodiments the combination ratio of hydroxamate-type and catecholate-type siderophores is 7:1. In some embodiments the combination ratio of hydroxamate-type and catecholate-type siderophores is 8:1. In some embodiments the combination ratio of hydroxamate-type and catecholate-type siderophores is 9:1. In some embodiments the combination ratio of hydroxamate-type and catecholate-type siderophores is 10:1. In some embodiments the combination ratio of hydroxamate-type and catecholate-type siderophores is 11:1. In some embodiments the combination ratio of hydroxamate-type and catecholate-type siderophores is 12:1. In some embodiments the combination ratio of hydroxamate-type and catecholate-type siderophores is 13:1. In some embodiments the combination ratio of hydroxamate-type and catecholate-type siderophores is 14:1. In some embodiments the combination ratio of hydroxamate-type and catecholate-type siderophores is 15:1. In some embodiments the combination ratio of hydroxamate-type and catecholate-type siderophores is 16:1. In some embodiments the combination ratio of hydroxamate-type and catecholate-type siderophores is 17:1. In some embodiments the combination ratio of hydroxamate-type and catecholate-type siderophores is 18:1. In some embodiments the combination ratio of hydroxamate-type and catecholate-type siderophores is 19:1. In some embodiments the combination ratio of hydroxamate-type and catecholate-type siderophores is 20:1.

The stoichiometric range for metal ion concentration relative to the siderophore concentration can vary from 1:1 to 1:10. In some embodiments, the stoichiometric range for metal ion concentration relative to siderophore concentration is at least 1:1. In some embodiments, the stoichiometric range for metal ion concentration relative to siderophore concentration is at least 1:2. In some embodiments, the stoichiometric range for metal ion concentration relative to siderophore concentration is at least 1:3. In some embodiments, the stoichiometric range for metal ion concentration relative to siderophore concentration is at least 1:4. In some embodiments, the stoichiometric range for metal ion concentration relative to siderophore concentration is at least 1:5. In some embodiments, the stoichiometric range for metal ion concentration relative to siderophore concentration is at least 1:6. In some embodiments, the stoichiometric range for metal ion concentration relative to siderophore concentration is at least 1:7. In some embodiments, the stoichiometric range for metal ion concentration relative to siderophore concentration is at least 1:8. In some embodiments, the stoichiometric range for metal ion concentration relative to siderophore concentration is at least 1:9. In some embodiments, the stoichiometric range for metal ion concentration relative to siderophore concentration is at least 1:10. In some embodiments, the stoichiometric range for metal ion concentration relative to siderophore concentration is 1:1. In some embodiments, the stoichiometric range for metal ion concentration relative to siderophore concentration is 1:2. In some embodiments, the stoichiometric range for metal ion concentration relative to siderophore concentration is 1:3. In some embodiments, the stoichiometric range for metal ion concentration relative to siderophore concentration is 1:4. In some embodiments, the stoichiometric range for metal ion concentration relative to siderophore concentration is 1:5. In some embodiments, the stoichiometric range for metal ion concentration relative to siderophore concentration is 1:6. In some embodiments, the stoichiometric range for metal ion concentration relative to siderophore concentration is 1:7. In some embodiments, the stoichiometric range for metal ion concentration relative to siderophore concentration is 1:8. In some embodiments, the stoichiometric range for metal ion concentration relative to siderophore concentration is 1:9. In some embodiments, the stoichiometric range for metal ion concentration relative to siderophore concentration is 1:10.

In some embodiments, disclosed herein are methods of making a photosynthetic power cell. In some embodiments, the method comprises forming a layer of artificial metallo-siderophores by combining (1) an amphiphilic siderophore (2) a metal ion and (3) a photosynthetic and/or photoresponsive microorganism. In some embodiments, the photosynthetic and photoresponsive microorganism is anchored to the amphiphilic siderophore. In some embodiments, the method further comprises adding the layer of artificial metallo-siderophores to the surface of a power cell.

In one embodiment, disclosed is a method of making a photosynthetic power cell, the method comprising combining an amphiphilic siderophore, a metal ion and a photosynthetic and/or photoresponsive microorganism, wherein the photosynthetic and/or photoresponsive microorganism is anchored to the amphiphilic siderophore.

In certain embodiments, the amphiphilic siderophore is a catecholate-type artificial siderophore or a hydroxamate-type artificial siderophore. For example the catecholate-type siderophore may comprise tri-catenated catecholate siderophore Syn-Enterobactin A, hexa-catenated catecholate Syn-Enterobactin B, Enterobactin or amphiphilic synthetic derivatives therefrom, Anguibactin or amphiphilic synthetic derivatives therefrom, Vanchrobactin or amphiphilic synthetic derivatives therefrom. Alternatively, the hydroxamate-type siderophore may comprise tri-substituted hydroxamate Syn-Ferrichrome A, hexa-substituted Syn-Ferrichrome B, Ferrichrome or amphiphilic synthetic derivatives therefrom, Desferrioxamine B or amphiphilic synthetic derivatives therefrom, Aerobactin or amphiphilic synthetic derivatives therefrom.

A variety of metal ions may be used. Further, as discussed herein, the stoichiometric range for metal ion concentration relative to the siderophore concentration can vary from 1:1 to 1:10 (and all ranges within). For example, in certain embodiments, the metal ion comprises at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr). For example, in certain embodiments, the metal ion is at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). In certain embodiments, the amphiphilic siderophore and the metal ion may bind to form a amphiphilic siderophore-metal ion coordinated complex. In some embodiments, a plurality of these amphiphilic siderophore-metal ion coordination complexes may exist. In some embodiments, the plurality of amphiphilic-metal ion coordination complexes may comprise at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr). In certain embodiments, the metal ion is at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Or, in other embodiments, the plurality of amphiphilic-metal ion coordination complexes may comprise a combination of any two of the at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Additionally or alternatively, the plurality of the amphiphilic-metal ion coordination complexes may comprise a combination of any three of the at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). In yet other embodiments, the plurality of the amphiphilic-metal ion coordination complexes may comprise a combination of all four of at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Or, for example, in yet other embodiments, other ions and stoichiometric ratios of the at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)) may be used.

In yet other embodiments, the amphiphilic siderophore-metal ion coordinated complex self-assembles with one or more additional amphiphilic siderophore-metal ion coordinated complexes of the same type to form a superstructure system. As used herein, a “superstructure system” is a structure comprising multiple units of a single structure. In some embodiments, the amphiphilic siderophore-metal ion coordinated complexes that self-assemble are the same amphiphilic siderophore-metal ion coordinated complexes. As used herein, “self-assembly” defines a process in which particles or materials spontaneously arrange predefined components into ordered superstructures. In some embodiments, one type of amphiphilic siderophore-metal ion coordinated complex may combine with a different type of amphiphilic siderophore-metal ion coordinated complex or several different types of amphiphilic siderophore-metal ion coordinated complexes. In some embodiments, the self-assembly is 1:1. In other embodiments, the self-assembly is 1:2. In other embodiments, the self-assembly is 2:1. In other embodiments, the self-assembly is 1:3. In other embodiments, the self-assembly is 3:1. Or, in yet other embodiments, a plurality of amphiphilic siderophore-metal ion coordinated complexes in other ratios may self-assemble.

In other embodiments, the superstructure system may be formed in a liquid-liquid interface through a process comprising directed self-assembly of the amphiphilic siderophore-metal ion coordinated complex. For example, to initiate metal-metal interactions within hydrophilic cores of the amphiphilic siderophore-metal ion coordinated complexes, while catalyzing hydrogen bonding, polar protic solvents, such as but not limited to, water, methanol, ethanol, and isopropanol and polar aprotic solvents, such as but not limited to, dichloromethane, acetonitrile, and acetone may be used. Non-polar solvents, such as but not limited to, chloroform, hexane, and 1,4-dioxane may be less effective solvents, as such solvents can induce hydrophobic π-π interactions within aromatic moieties. In an embodiment, using these non-covalent interactions at the polar- and non-polar interface with the siderophore may allow the formation of amphiphilic siderophore-metal ion coordinated complex assemblies and may provide a path to fabricate artificial siderophore superstructure systems in mesoscale. In other embodiments, the superstructure system may be formed in a liquid-liquid interface through a process comprising in-situ coordination programming of the amphiphilic siderophores with the one or more metal ions. In one embodiment, fabrication of superstructures via in-situ coordination with the one or more metal ions may follow directed self-assembly at the water/oil interface initializing metal ion coordination interactions with the amphiphilic siderophores followed by addition of a third solvent to catalyze the aggregation.

A variety of photosynthetic and/or photoresponsive microorganisms may be used. For example, in certain embodiments, the photosynthetic and/or photoresponsive microorganism is a photosynthetic and/or photoresponsive bacteria. For example, in certain embodiments, the bacteria may comprise a marine purple non-sulfur bacteria. Such bacteria may comprise Rhodobacter sphaeroides, Rhodobacter capsulatus or other types of marine purple bacteria. Additionally and/or alternatively, the bacteria may comprise a cyanobacteria. For example, in certain embodiments, cyanobacteria from the Synechocystis genus, as for example, Synechocystis sp. FCC 6803, may be used.

### Compositions for Biophotonic Charge Storages

In yet other embodiments, disclosed are compositions for biophotonic charge storage. The compositions may comprise an amphiphilic siderophore, a metal ion, and a photosynthetic and/or photoresponsive microorganism, wherein the photosynthetic and/or photoresponsive microorganism is anchored to the amphiphilic siderophore.

In certain embodiments, the amphiphilic siderophore is a catecholate-type artificial siderophore or a hydroxamate-type artificial siderophore. For example the catecholate type siderophore may comprise tri-catenated catecholate Syn-Enterobactin A, hexa-catenated catecholate Syn-Enterobactin B, Enterobactin or amphiphilic synthetic derivatives therefrom, Anguibactin or amphiphilic synthetic derivatives therefrom, Vanchrobactin or amphiphilic synthetic derivatives therefrom. Alternatively, the hydroxamate type siderophore may comprise tri-substituted hydroxamate Syn-Ferrichrome A, hexa-substituted Syn-Ferrichrome B, Ferrichrome or amphiphilic synthetic derivatives therefrom, Desferrioxamine B or amphiphilic synthetic derivatives therefrom, Aerobactin or amphiphilic synthetic derivatives therefrom. Or other catecholate-type artificial siderophores or hydroxamate-type artificial siderophores may be used.

A variety of metal ions may be used. Further, as discussed herein, the stoichiometric range for metal ion concentration relative to the siderophore concentration can vary from 1:1 to 1:10 (and all ranges within). For example, in certain embodiments, the metal ion comprises at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr). In certain embodiments, the metal ion is at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)).

In certain embodiments, the amphiphilic siderophore and the one or more metal ion may bind to form a amphiphilic siderophore-metal ion coordinated complex. In some embodiments, a plurality of these amphiphilic siderophore-metal ion coordination complexes may be used. In some embodiments, the plurality of amphiphilic-metal ion coordination complexes may comprise at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr). For example, in certain embodiments, the metal ion is an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), and/or an actinide (e.g., Thorium (Th), Protactinium (Pa)). Or, in other embodiments, the plurality of amphiphilic-metal ion coordination complexes may comprise a combination of any two of the at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Or, in yet other embodiments, the plurality of the amphiphilic-metal ion coordination complexes may comprise a combination of any three of the at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Additionally and/or alternatively, the plurality of the amphiphilic-metal ion coordination complexes may comprise a combination of all four of at least one of an alkaline-earth metal ion (e.g., magnesium (Mg), calcium (Ca)), a transition metal ion (e.g., iron (Fe), zinc (Zn), copper (Cu), nickel (Ni)), a lanthanide (e.g., Cerium (Ce), Neodymium (Nd)), or an actinide (e.g., Thorium (Th), Protactinium (Pa)). Or, in other embodiments, other ions and/or stoichiometric ratios may be used.

In yet other embodiments, the amphiphilic siderophore-metal ion coordinated complex self-assembles with one or more amphiphilic siderophore-metal ion coordinated complexes to form a superstructure system. In some embodiments, the amphiphilic siderophore-metal ion coordinated complexes that self-assemble are the same amphiphilic siderophore-metal ion coordinated complexes (i.e., a siderophore superstructure) Or, one type of amphiphilic siderophore-metal ion coordinated complex may combine with a different type of amphiphilic siderophore-metal ion coordinated complex or several different types of amphiphilic siderophore-metal ion coordinated complexes. In some embodiments, the self-assembly is 1:1. In other embodiments, the self-assembly is 1:2. In other embodiments, the self-assembly is 2:1. In other embodiments, the self-assembly is 1:3. In other embodiments, the self-assembly is 3:1. Or, in yet other embodiments, a plurality of amphiphilic siderophore-metal ion coordinated complexes of other ratios may self-assemble.

In other embodiments, the superstructure system may be formed in a liquid-liquid interface through a process comprising directed self-assembly of the amphiphilic siderophore-metal ion coordinated complex. For example, to initiate metal-metal interactions within hydrophilic cores of the amphiphilic siderophore-metal ion coordinated complexes, while catalyzing hydrogen bonding, polar protic solvents, such as water, methanol, ethanol, and isopropanol and polar aprotic solvents such as dichloromethane, acetonitrile, and acetone may be tested to use as good solvents. Non-polar solvents, such as chloroform, hexane, and 1,4-dioxane may be tested as less preferred solvents, as these solvents may induce hydrophobic 7t-7t interactions within aromatic moieties. Understanding these non-covalent interactions at the polar- and non-polar interface with the siderophore may reveal the formation of amphiphilic siderophore-metal ion coordinated complex assemblies and may provide a path to fabricate artificial siderophore superstructure systems in mesoscale.

In other embodiments, the superstructure system may be formed in a liquid-liquid interface through a process comprising in-situ coordination programming of the amphiphilic siderophores with the one or more metal ions. In one embodiment, fabrication of superstructures via in-situ coordination with the one or more metal ions may follow directed self-assembly at the water/oil interface initializing metal ion coordination interactions with the amphiphilic siderophores followed by addition of a third solvent to catalyze the aggregation.

A variety of photosynthetic and/or photoresponsive microorganisms may be used. For example, in certain embodiments, the photosynthetic and/or photoresponsive microorganism is a photosynthetic and/or photoresponsive bacteria. For example, in certain embodiments, the bacteria may comprise a marine purple non-sulfur bacteria. Such bacteria may comprise Rhodobacter sphaeroides, Rhodobacter capsulatus or other types of marine purple bacteria. Additionally and/or alternatively, the bacteria may comprise a cyanobacteria. For example, in certain embodiments, cyanobacteria from the Synechocystis genus, for example, Synechocystis sp. FCC 6803, may be used.

### Devices for Biophotonic Charge Storage

In other embodiments, disclosed are devices for biophotonic charge storage. The devices may, in alternate embodiments, comprise any one, or a plurality of, each of the embodiments of the compositions disclosed herein. Also, the devices may be produced at least in part using the methods disclosed herein. In certain embodiments, the device may comprise a layer of a siderophore-metal ion coordinated complex with a photosynthetic and photoresponsive microorganism, wherein the siderophore layer is placed between an anode and a cathode. In certain embodiments, a single layer of a siderophore-metal ion coordinated complex with a photosynthetic and photoresponsive microorganism is used. Or in some cases, a plurality of siderophore layers may be used.

In some embodiments, a plurality of siderophore layers my include 2 or more layers, 3 or more layers, 4 or more layers, 5 or more layers, 6 or more layers, 7 or more layers, 8 or more layers, 9 or more layers, 10 or more layers, 11 or more layers, 12 or more layers, 13 or more layers, 14 or more layers, 15 or more layers, 16 or more layers, 17 or more layers, 18 or more layers, 19 or more layers, or at least 20 layers. In some embodiments, at least 20 layers is inclusive of 20 layers and extends to at least 100 layers. In some embodiments, a plurality of siderophore layers may be at least 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 layers.

A variety of photosynthetic and/or photoresponsive microorganisms may be used in the device. For example, in certain embodiments, the photosynthetic and/or photoresponsive microorganism is a photosynthetic and/or photoresponsive bacteria. For example, in certain embodiments, the bacteria may comprise a marine purple non-sulfur bacteria. Such bacteria may comprise Rhodobacter sphaeroides, Rhodobacter capsulatus or other types of marine purple bacteria. Additionally and/or alternatively, the bacteria may comprise a cyanobacteria. For example, in certain embodiments, cyanobacteria from the Synechocystis genus, for example, Synechocystis sp. FCC 6803, may be used.

In some embodiments, the siderophore is anchored to the bacteria by selective recognition of receptor proteins on the cell membrane of microbes. In some embodiments, the receptor proteins comprise c-Cyts of MtrC and OmcA. Or, the ancoring may rely on other membrane protein and/or receptors interactions.

In some embodiments, the single siderophore layer is placed between an anode and a cathode, wherein the initial photovoltage achieved is at least 1.0 V and a capacitance of at least 0.5 F/m², or at least 3.0 V and a capacitance of at least 2.0 F/m². In some embodiments, higher levels or ranges within these ranges may be achieved. For example, In some embodiments, the initial photovoltage achieved is at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.4, 3.5, 3.6, 3.7, 3.8, 3.9, 4.0, 4.1, 4.2, 4.3, 4.4, 4.5, 4.6, 4.7, 4.8, 4.9, or 5.0 V. In some embodiments, the initial capacitance achieved is at least 0.5, 0.6, 0.7, 0.8, 0.9, 1.0, 1.1, 1.2, 1.3, 1.4, 1.5, 1.6, 1.7, 1.8, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 F/m².

In some embodiments, the siderophore layer is placed between an anode and a cathode, wherein the anode is indium tin oxide. In other embodiments, the anode may be indium molybdenum oxide, or other doped binary compounds such as aluminum-doped zinc oxide (AZO) and indium-doped cadmium oxide. In other embodiments, the anode may be gallium or indium-doped zinc oxide (GZO or IZO), or tungsten oxide and molybdenum oxide may be used. Or, other materials may be used for the anode including rare earth oxides. In some embodiments, the siderophore layer may be a single siderophore layer. In some embodiments, the siderophore layer may be a plurality of siderophore layers. In some embodiments, a plurality of siderophore layers may be more than one siderophore layer. In some embodiments, a plurality of siderophore layers may be at least five siderophore layers. In some embodiments, a plurality of siderophore layers may be at least 10, 15, 20, 25, 30, 40, 45, 50, 55, 60, 65, 70, 75, 80, 85, 90, 95, or 100 siderophore layers.

In some embodiments, the single siderophore layer is placed between an anode and a cathode, wherein the cathode is n-Si, or aluminum, or gold, or silver. Additionally and/or alternatively, in some embodiments, the biophotonic power cell can be constructed by patterning three trenches, each with dimensions of at least 10 μm in length, 5 μm in width and 200 nm in depth on a pre-cleaned n-doped silicon wafer by focused ion-beam lithography (FIB), or at least 50 m in length, 20 μm in width and 500 nm in depth. In some embodiments, the three trenches may have dimensions between 10 μm and 50 μm in length, 5 μm and 20 μm in width, and 200 nm and 500 nm in depth. In some embodiments, the three trenches, each have a dimension of at least 10, 15, 20, 25, 30, 35, 40, 45, or 50 μm in length. In some embodiments, the three trenches each have a dimension of at least 5, 10, 15, or 20 μm in width. In some embodiments, the three trenches each have a dimension of at least 200, 210, 220, 230, 240, 250, 260, 270, 280, 290, 300, 310, 320, 330, 340, 350, 360, 370, 380, 390, 400, 410 420, 430, 440, 450, 460, 470, 480, 490, or 500 nm in depth.

In some embodiments, the probe current will be kept at least at 10 kV to at least 30 kV or a current appropriate to reach the targeted depth. In some embodiments, the probe current will be kept at least at 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 40, 41, 42, 43, 44, 45, 46, 47, 48, or 49 kV.

In some embodiments, a negative photoresist may be deposited to fill the patterned lines with at least 100 nm thick photoresist layer or at least 1 μm photoresist layer. In some embodiments, the photoresist layer may be between 100 nm thick or 1000 nm thick. In some embodiments, the photoresist layer may be at least 110 nm thick. In some embodiments, the photoresist layer may be at least 120 nm thick. In some embodiments, the photoresist layer may be at least 130 nm thick. In some embodiments, the photoresist layer may be at least 140 nm thick. In some embodiments, the photoresist layer may be at least 150 nm thick. In some embodiments, the photoresist layer may be at least 160 nm thick. In some embodiments, the photoresist layer may be at least 170 nm thick. In some embodiments, the photoresist layer may be at least 180 nm thick. In some embodiments, the photoresist layer may be at least 190 nm thick. In some embodiments, the photoresist layer may be at least 200 nm thick. In some embodiments, the photoresist layer may be at least 210 nm thick. In some embodiments, the photoresist layer may be at least 220 nm thick. In some embodiments, the photoresist layer may be at least 230 nm thick. In some embodiments, the photoresist layer may be at least 240 nm thick. In some embodiments, the photoresist layer may be at least 250 nm thick. In some embodiments, the photoresist layer may be at least 260 nm thick. In some embodiments, the photoresist layer may be at least 270 nm thick. In some embodiments, the photoresist layer may be at least 280 nm thick. In some embodiments, the photoresist layer may be at least 290 nm thick. In some embodiments, the photoresist layer may be at least 300 nm thick. In some embodiments, the photoresist layer may be at least 310 nm thick. In some embodiments, the photoresist layer may be at least 320 nm thick. In some embodiments, the photoresist layer may be at least 330 nm thick. In some embodiments, the photoresist layer may be at least 340 nm thick. In some embodiments, the photoresist layer may be at least 350 nm thick. In some embodiments, the photoresist layer may be at least 360 nm thick. In some embodiments, the photoresist layer may be at least 370 nm thick. In some embodiments, the photoresist layer may be at least 380 nm thick. In some embodiments, the photoresist layer may be at least 390 nm thick. In some embodiments, the photoresist layer may be at least 400 nm thick. In some embodiments, the photoresist layer may be at least 410 nm thick. In some embodiments, the photoresist layer may be at least 420 nm thick. In some embodiments, the photoresist layer may be at least 430 nm thick. In some embodiments, the photoresist layer may be at least 440 nm thick. In some embodiments, the photoresist layer may be at least 450 nm thick. In some embodiments, the photoresist layer may be at least 460 nm thick. In some embodiments, the photoresist layer may be at least 470 nm thick. In some embodiments, the photoresist layer may be at least 480 nm thick. In some embodiments, the photoresist layer may be at least 490 nm thick. In some embodiments, the photoresist layer may be at least 500 nm thick. In some embodiments, the photoresist layer may be at least 510 nm thick. In some embodiments, the photoresist layer may be at least 520 nm thick. In some embodiments, the photoresist layer may be at least 530 nm thick. In some embodiments, the photoresist layer may be at least 540 nm thick. In some embodiments, the photoresist layer may be at least 550 nm thick. In some embodiments, the photoresist layer may be at least 560 nm thick. In some embodiments, the photoresist layer may be at least 570 nm thick. In some embodiments, the photoresist layer may be at least 580 nm thick. In some embodiments, the photoresist layer may be at least 590 nm thick. In some embodiments, the photoresist layer may be at least 600 nm thick. In some embodiments, the photoresist layer may be at least 610 nm thick. In some embodiments, the photoresist layer may be at least 620 nm thick. In some embodiments, the photoresist layer may be at least 630 nm thick. In some embodiments, the photoresist layer may be at least 640 nm thick. In some embodiments, the photoresist layer may be at least 650 nm thick. In some embodiments, the photoresist layer may be at least 660 nm thick. In some embodiments, the photoresist layer may be at least 670 nm thick. In some embodiments, the photoresist layer may be at least 680 nm thick. In some embodiments, the photoresist layer may be at least 690 nm thick. In some embodiments, the photoresist layer may be at least 700 nm thick. In some embodiments, the photoresist layer may be at least 710 nm thick. In some embodiments, the photoresist layer may be at least 720 nm thick. In some embodiments, the photoresist layer may be at least 730 nm thick. In some embodiments, the photoresist layer may be at least 740 nm thick. In some embodiments, the photoresist layer may be at least 750 nm thick. In some embodiments, the photoresist layer may be at least 760 nm thick. In some embodiments, the photoresist layer may be at least 770 nm thick. In some embodiments, the photoresist layer may be at least 780 nm thick. In some embodiments, the photoresist layer may be at least 790 nm thick. In some embodiments, the photoresist layer may be at least 800 nm thick. In some embodiments, the photoresist layer may be at least 810 nm thick. In some embodiments, the photoresist layer may be at least 820 nm thick. In some embodiments, the photoresist layer may be at least 830 nm thick. In some embodiments, the photoresist layer may be at least 840 nm thick. In some embodiments, the photoresist layer may be at least 850 nm thick. In some embodiments, the photoresist layer may be at least 860 nm thick. In some embodiments, the photoresist layer may be at least 870 nm thick. In some embodiments, the photoresist layer may be at least 880 nm thick. In some embodiments, the photoresist layer may be at least 890 nm thick. In some embodiments, the photoresist layer may be at least 900 nm thick. In some embodiments, the photoresist layer may be at least 910 nm thick. In some embodiments, the photoresist layer may be at least 920 nm thick. In some embodiments, the photoresist layer may be at least 930 nm thick. In some embodiments, the photoresist layer may be at least 940 nm thick. In some embodiments, the photoresist layer may be at least 950 nm thick. In some embodiments, the photoresist layer may be at least 960 nm thick. In some embodiments, the photoresist layer may be at least 970 nm thick. In some embodiments, the photoresist layer may be at least 980 nm thick. In some embodiments, the photoresist layer may be at least 990 nm thick. In some embodiments, the photoresist layer may be at least 1000 nm thick. Also, in some embodiments, both ends of the resist can be etched by exposing the negative resist to UV light.

In other embodiments, amorphous silica (SiO₂) can be deposited as a dielectric to avoid the short circuit issues between two electrode materials, followed by etching the remaining portion of the negative resist. The biophotonic layer of choice can then be drop-casted into each patterned trench. The substrate power cell prepared in this manner can be partially vacuum-dried prior to deposit of the transparent indium tin oxide (ITO) layer. The power cell may then be sealed and stored.

In some embodiments, the biophotonic power cell can be exposed to simulated sunlight at an intensity of at least 100 mWcm⁻² approximating to 1 sun illumination using the solar simulator over at least 100 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 200 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 300 s as the first charging duration to at least 1 hr as the first charging duration, under open circuit conditions. In some embodiments, the biophotonic power cell can be exposed to simulated sunlight at an intensity of at least 100 mWcm² approximating to 1 sun illumination using the solar simulator over at least 400 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 500 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 600 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 700 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 800 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 900 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 1000 s as the first charging duration to at least 1 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 100 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 200 s as the first charging duration to at least 2 hr as the first charging duration, under open circuit conditions. In some embodiments, the biophotonic power cell can be exposed to simulated sunlight at an intensity of at least 100 mWcm² approximating to 1 sun illumination using the solar simulator over at least 300 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 400 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 500 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 600 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 700 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 800 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 900 s as the first charging duration to at least 2 hr as the first charging duration, under open circuit conditions. In some embodiments, the biophotonic power cell can be exposed to simulated sunlight at an intensity of at least 100 mWcm⁻² approximating to 1 sun illumination using the solar simulator over at least 1000 s as the first charging duration to at least 2 hr as the first charging duration, under open circuit conditions.

In yet other embodiments, charge-discharge responses may be obtained over a 0 to 1 V voltage window at different applied current starting from 10 μA/cm² to 1 mA/cm², to obtain the optimal capacitance. From the galvanostatic charge-discharge response with respect to the applied current range, the capacitance can be calculated from the active area and the slope of the discharge curve. In various embodiments, the devices provide a reproducibility and accuracy threshold of >98%.

### EXAMPLES

The disclosure may be better understood by reference to the following non-limiting examples.

### Example 1—Synthesis of Artificial Siderophores

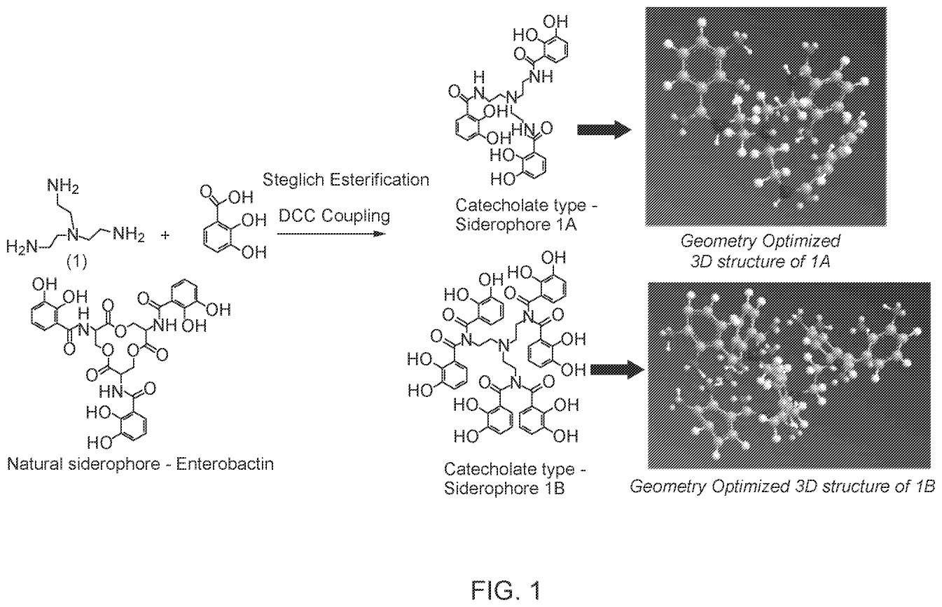

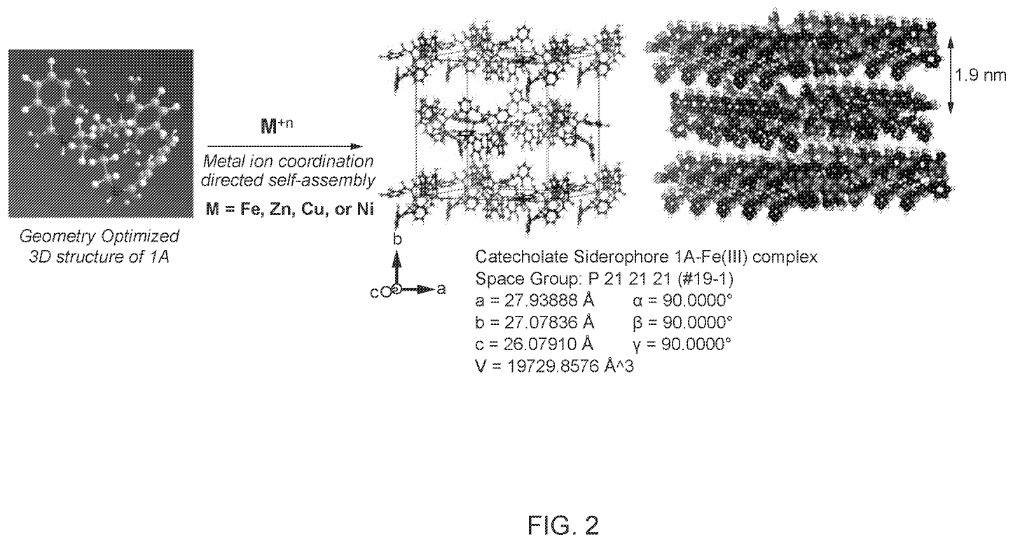

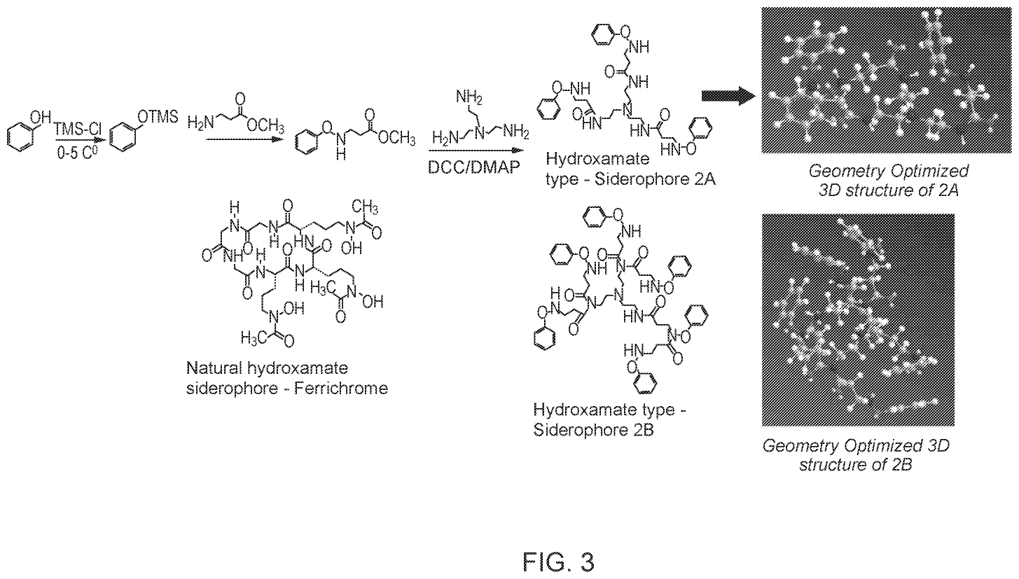

Catecholate type artificial siderophores Tri- and hexa-catenated catecholate type two artificial siderophores were designed by augmenting an enterobactin natural siderophore by replacing the tri-carboxylate tether of the enterobactin with a tripod amine tether as depicted in FIG. 1. Starting from the tripod amine tether, N,N′-bis-(2-aminoethyl)ethane-1,2-diamine (1), tri and hexa-catenated catecholate Syn-Enterobactin A and Syn-Enterobactin B were synthesized by reacting the enterobactin and tripod amine tether with 2,3-dihydroxybenzoic acid using Steglich esterification. The catenation was regulated by controlling the molar ratio of tripod amine precursor and 2,3-dihydroxybenzoic acid. The crystal structures were deduced and constructed. Their respective analogues of coordination complexes (FIG. 2) can be prepared by reacting the siderophores (i.e., 1A and 1B) with respective metal ionprecursors and structures fully characterized, including their crystal structures and packing patterns in the solid state. The coordination complex formation between each respective metal ion and catecholate units leads to the coordination geometrical changes, leading to self-assemblies with different morphologies. The metal-coordinated complexes can be produced by coordinating the metal to hydroxy groups of two catecholate units and amide carbonyl and amine groups, resulting in hexacoordinated octahedral geometry at the metal coordination center (FIG. 3). An octahedral geometry for a catecholate siderophore-Fe(III) complex was previously reported as a model compound to an Enterobactin analogue.

Hydroxamate type artificial siderophores—Two new hydroxamate type artificial siderophores were designed as model compounds for a natural siderophore, ferrichrome secreted from Ustilago sphaerogena (fungi). Starting from phenol and N,N-bis-(2-aminoethyl)ethane-1,2-diamine, the tri- and hexa-substituted hydroxamate Syn-Ferrichrome A and Syn-Ferrichrome B were synthesized. As depicted in FIG. 3, the method included the steps of silylating the hydroxy functionality of phenol with trimethylsilylchloride at 0 to 5° C. in anhydrous tetrahydrofuran (THF), followed by reacting with methyl β-alanine. The intermediate precursor, methyl-3-(phenoxyamino)propionate was synthesized and fully characterized using FTIR, NMR, and elemental analysis prior to reacting with the tripod amine tether, N,N-bis-(2-aminoethyl)ethane-1,2-diamine. By controlling the molar ratio of the intermediate precursor, methyl-3-(phenoxyamino)propionate and tripod amine tether, at 3.1 and 6:1, siderophore 1 and 2 were synthesized using Steglich esterification. The final structures were characterized using FT-IR and ¹³C and ¹H-NMR. Crystallization can be performed, and single crystal structures deduced by using single crystal and powder XRD combined with HR-TEM and Selective Area diffraction analysis. (FIG. 3).

### Example 2—Synthesis of Analogues of Coordinating Complexes Using the Artificial Siderophores

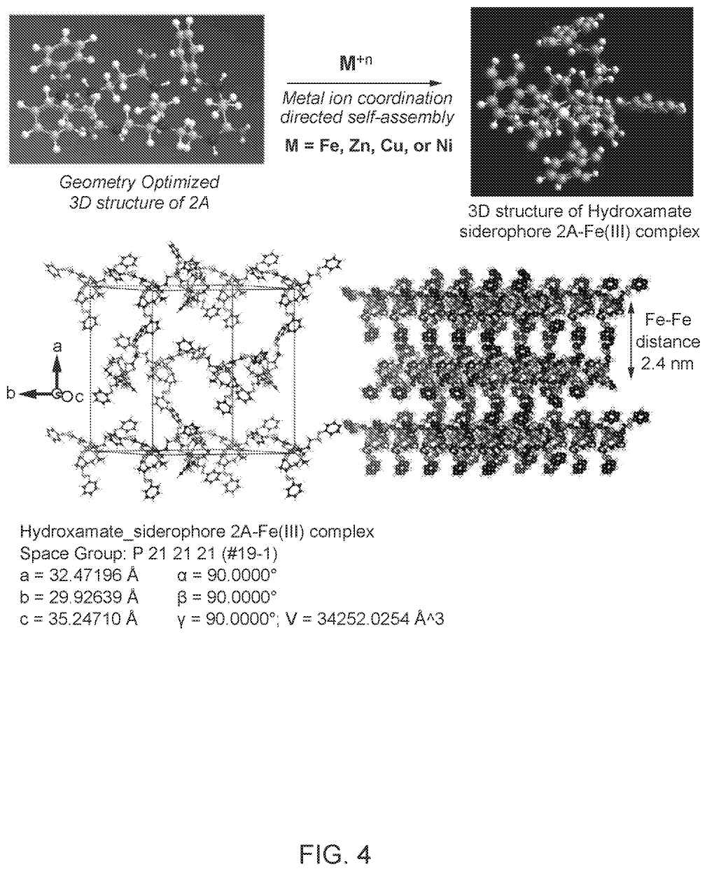

The analogues of coordination complexes for hydroxamate and catecholate siderophores (FIG. 4) were synthesized by mixing each hydroxamate Syn-Ferrichrome A and Syn-Ferrichrome B with respective ion precursor either in the form of chloride or acetate by stirring at room temperature. The purification and crystallization steps were conducted to obtain highly pure crystals of each siderophore-metal ion complexes. A previously reported ferrichrome artificial hydroxamate siderophore complex revealed that a metal ion coordinated to amide carbonyl groups and nitro oxide groups forming hexacoordinated octahedral geometry. Similarly, hydroxamate Syn-Ferrichrome A forms hexacoordinated Fe(III) complex by coordinating to amide carbonyl and amine groups of the central tripod amide unit and amine groups of two β-alanine units, resulting in octahedral geometry at the metal ion center (FIG. 4).

### Example 3—Creating a Superstructure System of Artificial Siderophores Through Liquid-Liquid Interface Self-Assembly Processes

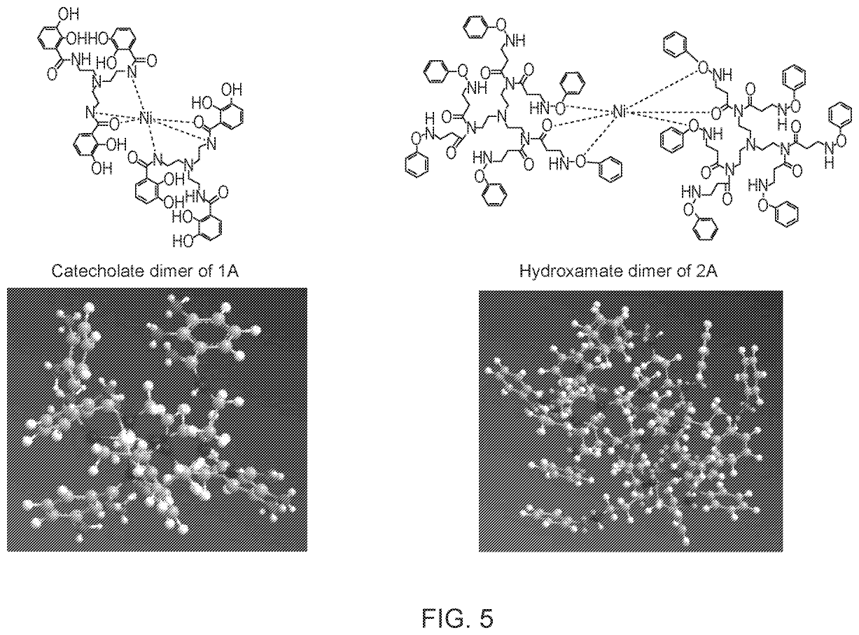

Hydroxamate and catecholate siderophores can lead to the dimer formation via coordination with one or more metal ions. Additionally, coordination directed self-assembly can produce superstructure systems of dimers and/or mixed combinations (FIG. 5).

This example provides a liquid-liquid interface (LLI) directed self-assembly approach to create superstructures of the artificial siderophores. There are no prior LLI self-assembly approaches developed for coordination programing of either natural or artificial siderophores. The interface between two immiscible liquids can provide an important alternative path for the self-assembly and chemical manipulation of molecular building blocks and nanocrystals.

Utilizing all available non-covalent interactions and hydrophilic and hydrophobic functional units in catecholate and hydroxamate type artificial siderophores synthesized in this example, two new LLI self-assembly fabrication processes can be developed. The first fabrication process follows directed self-assembly of siderophores-metal ion coordinated complexes at the LLI and the second approach follows the in-situ coordination programming (“coordinate andgo”) of the siderophores with metal ions at the LLI. These two approaches are described below.

### 1. Directed Self-Assembly Approach to Fabricate Siderophore-Metal Ion Complexes at the LLI

This method utilizes a process of self-assembling metal ion coordinated siderophore complexes at the LLI creating a nonhomogeneous interface using two immiscible solvents (oil-in water system) where one solvent is a good solvent for the complex and one solvent is a poor solvent for the complex. The solvent polarity, dielectric nature, acidity and basicity, and possible interfacial interactions with the siderophore's functional groups are considered for the solvent selection. For example, hydrophilic and hydrophobic interactions of the functional groups, π-π interactions and/or hydrogen bonding (in Syn-Enterobactin A) optionally can be used to make superstructures of metal-coordinated siderophores.

To initiate metal-metal interactions within hydrophilic cores, while catalyzing hydrogen bonding, polar protic solvents, such as water, methanol, ethanol, and isopropanol and polar aprotic solvents such as dichloromethane, acetonitrile, and acetone are tested to use as good solvents. Non-polar solvents, such as chloroform, hexane, and 1,4-dioxane, that induce hydrophobic π-π interactions within aromatic moieties are tested as poor solvents. Understanding these non-covalent interactions at the polar- and non-polar interface with the siderophore can reveal the formation of siderophore-metal ion complex assemblies and provide paths to fabricate artificial siderophore superstructure systems in mesoscale.

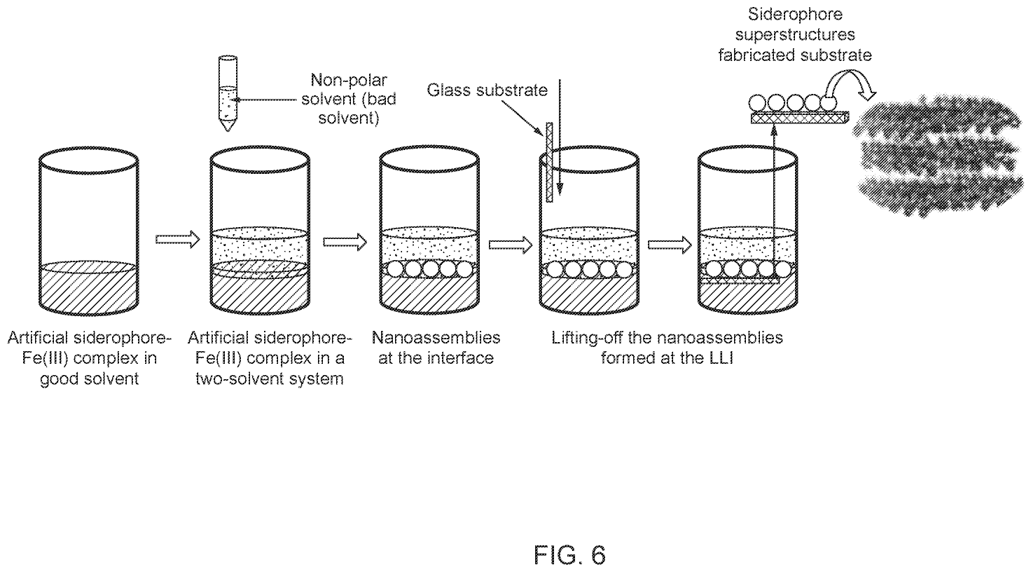

Fabrication process: A typical metal ion coordinated siderophore superstructure fabrication process using the directed assembly at the LLI may follow the fabrication steps illustrated in FIG. 6. In the first step, metal ion-coordinated siderophore is dissolved in the selected polar protic or polar aprotic solvent (e.g., 25 mg siderophore dissolved in about 2 mL good solvent). In a second step, equal volume of non-polar solvent of choice is added slowly into the solution, creating oil-in-water interface system. The tightly capped solution is allowed to stand 24 to 48 hours or until the aggregative assemblies formed at the interface of two solvents. By slowly inserting a fabrication glass substrate, which is plasma cleaned and soaked 24 hours in the good solvent the film comprising the siderophore superstructure can be lifted off from the side and onto the substrate.

2. In-Situ Coordination Programming-Directed Self-Assembly of Siderophores with Metal Ions at the LLI

Initializing the metal ion coordination to catecholate hydroxy functional groups, amine, and amide carbonyl functional groups in catecholate type Syn-Enterobactin A-Syn-Enterobactin B and hydroxamate type Syn-Ferrichrome A-Syn-Ferrichrome B, in-situ interfacial programming of siderophores at the LLI can be demonstrated. Thus, the method may first dissolve the siderophore in a polar protic solvent (good solvent), thereby catalyzing hydrogen bonding interactions and solvent-solute interactions. Augmenting a water-oil interface approach followed by aggregation by adding a third non-polar solvent, the superstructures of siderophore-metal ion coordinated complexes can be fabricated at the LL.

Fabrication process: As illustrated in FIG. 6, fabrication of superstructures via in-situ coordination with one or more metal ions follows directed self-assembly at the water/oil interface initializing metal ion coordination interactions with the siderophores followed by addition of a third solvent to catalyze the aggregation. In the first step, a siderophore dissolves (25 mg) in the selected polar protic or polar aprotic solvent (2 mL, good, i.e., polar, solvent) and adds slowly into an aqueous solution of one or more metal ion in an equal volume, while maintaining the molar ratio between siderophore to metal ions at 1:1. The tightly capped solution is allowed to stand 24 to 48 hours for complexation at the interface of two solvents. To initiate the aggregation process, a poor (i.e., non-polar) solvent is added slowly and allowed to let stand another 24 to 48 hours. Then by slowly inserting the fabrication glass substrate, which is plasma cleaned and soaked 24 hours in the same polar protic solvent used to dissolve the siderophore, the film can be lifted off (from the side) onto the substrate.

### Example 4—Fabrication and Performance Evaluation of the Biophotonic Power Cell Prototype

### Method of Fabrication

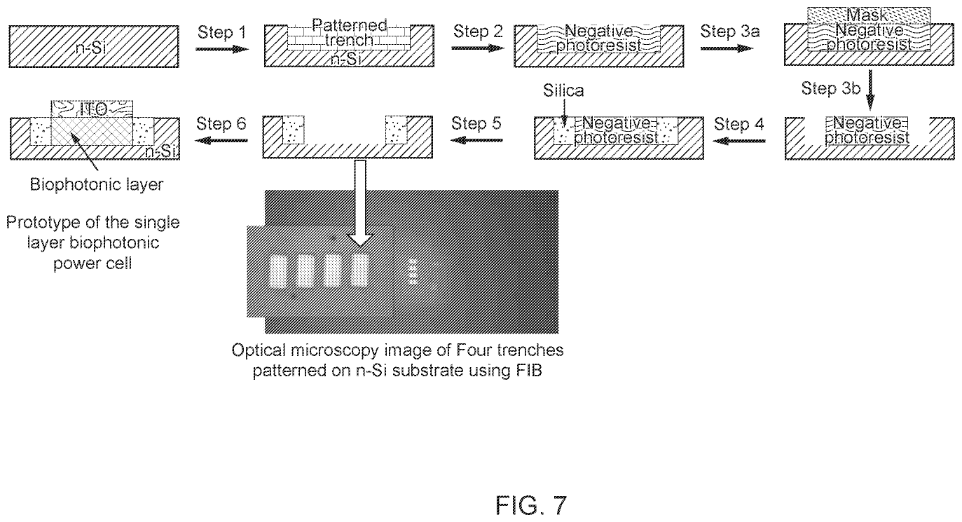

A biophotonic power cell can be constructed following a single layer design with the power cell configuration of ITO/photosynthetic layer/n-Si. Each power cell can have three devices. As depicted in FIG. 7, the process flow for the fabrication of the prototype can follow patterning several (e.g., three) trenches, each with dimension of 10 μm in length, 5 μm in width and 200 nm in depth, on a pre-cleaned n-doped silicon wafer by focused ion-beam lithography (FIB). To meet the targeted depth, the probe current can be kept at 30 kV. In the second step, a negative photoresist can be deposited to fill the patterned lines with 200 nm thick photoresist layer. Exposing the negative resist to UV light, both ends of the resist (˜2 μm from each side of each trench) can be etched using a patterned mask in the step 3. In the consecutive steps, the substrate can be prepared to deposit the biophotonic layer by first depositing amorphous silica (SiO₂) as dielectrics to avoid the short circuit issues between two electrode materials, followed by etching the remaining portion of the negative resist. The most optimized biophotonic layer can then be drop casted (˜ 6 μL in to each well) into each patterned trench. The substrate power cell prepared in this manner can then be partially vacuum dried prior to deposit the transparent ITO layer. Next, the power cell can be sealed and stored in dark overnight prior to begin collecting the measurements by exposing to sunlight.

### Method of Optimization of the Photoresponse of the Power Cell

The fabricated biophotonic power cell can be evaluated as for example, by exposing the cell to simulated sunlight at an intensity of 100 mWcm⁻² approximating to 1 sun illumination using the solar simulator over 200 s (optimized parameters from milestone 1) as the first charging duration, under open circuit conditions. The photovoltage can be recorded in the dark to determine the discharging time. By varying the charging time (exposure to sunlight), photovoltage can be recorded for minimum of five charge-discharge cycles. The data can be correlated with a state-of-the art biophotonic power cell, which constructed from photosynthetic bacteria isolated from R. sphaeroides that yield photovoltage of 0.45 V with longest discharge time 26 min for a charging time of ˜ 14 min. These performance measuring parameters (exposure time and thickness of the photoactive layer (depth of the trench)) are optimized tomeet a power cell photovoltage threshold of 0.5 V with effective charge storage over 15 minutes.

### Method of Evaluating the Power Cell Capacitance

The extent of charge accumulation by the purple bacteria in the power cell under different exposure time is determined by its light-harvesting capacity and photochemical activity rather than its innate charge storage capacity. As a result, the discharge times with respect to different exposure time do not necessarily convey the relative charge storage capacity of the power cell as each it will be charged to different extent under illumination. To investigate the capacitance of the prototype at a macroscopic level, galvanostatic charge-discharge measurements and CV can be performed. Near-symmetrical charge-discharge responses can be obtained over a 0 to 1 V voltage window at a different applied current starting from 10 μA/cm² to 1 mA/cm², to obtain the optimal capacitance, which surpasses the threshold. From the galvanostatic charge-discharge response with respect to the applied current range, the capacitance can be calculated from the active area and the slope of the discharge curve. Three replicates of measurements will be collected to reach the reproducibility and accuracy threshold of >98%.

### Example 5—Feasibility Assessment and Performance Validity of the Biophotonic Power Cell to Validate its Utility for Powering a Biosensor

To assess the ability of the prototype to power a small electronic device, such as a biosensor, tests are conducted to determine whether the designed power cell can power a commercial blood glucose meter (One Touch, 3V power input). This test allows direct comparison with current research and developments where conventional biophotonic devices have been shown to power small electronic devices, such as LEDs and digital clocks. Since biosensors often work over short measuring periods, interspaced by longer periods of inactivity, powering a hand-held blood glucose meter can be selected as a potential application.

### Designing the Electrical Circuit

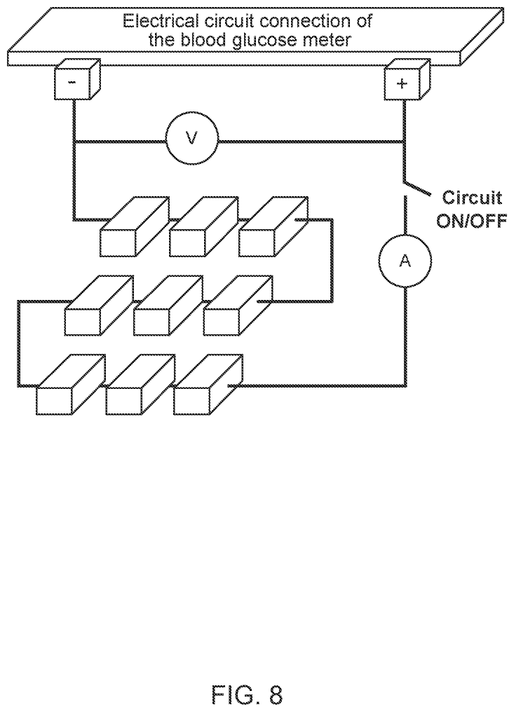

The electrical circuit design shown in the FIG. 8 can be constructed by connecting an array of nine biophotonic cells in three parallel clusters and each cell in a cluster will connect in series. A pulse generation circuit can be placed between the glucose meter battery connection and the biophotonic power cell array. Also, a voltmeter and an ammeter can be connected to the circuit to measure the potential (V) and the current (A), respectively.

### Powering A Blood Glucose Meter

The biophotonic power cell may be used for a variety of applications. For example, to power a blood glucose meter, an array of nine cells should produce 3V. Thus, to accumulate the required photovoltage, the power cell array is charged for about an hour. Then, the circuit is closed for 1 minute (initial attempt) during, which the glucose meter will be pulsed at a frequency of one pulse every 2.5 s. Then the number of average of flashes produced in 1 minute period is determined (e.g., n=10 tests), to confirm that the biophotonic power cell array generates the requisite bursts of power sufficient to power the glucose meter. Optimized parameters for duration of charging the power cell array are identified, and circuit ON and OFF time for keeping the biosensor on for a longer duration.

### Example 6: Photocurrent Generation in Cyanobacteria-Metallo-Surfactant-Based Bio-Photovoltaics

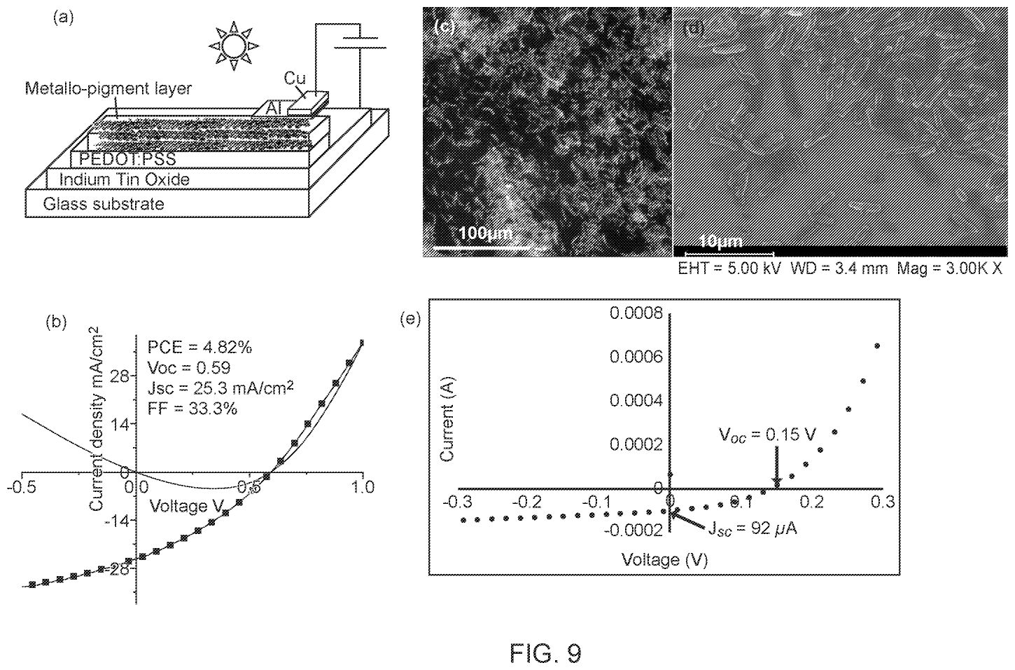

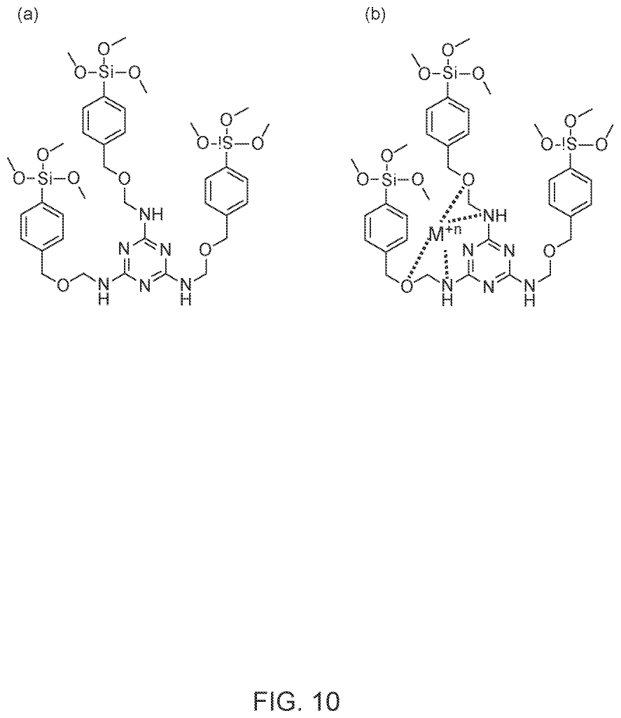

Fe(III)-coordinated artificial siderophores were fabricated to form superstructures-anchored metallo-pigmented organic molecules and cyanobacteria for use as light harvesters to capture sunlight for electric charge generation. These synthetic light harvesters were used to develop a single layer photovoltaic device (FIG. 9a), which was able to generate a photocurrent as high as 25 mA/cm² with power conversion efficiency of 4.82% (FIG. 9b). The device structure was optimized with an active layer thickness of 100-180 nm and a metallo-dye load of 75% (w/w %) with respect to the load of the Fe(III)-coordinated artificial siderophore. The Fe(III)-coordinated artificial siderophores were fabricated using methyl melamine-silane (FIG. 10a). The methyl-melamine-silane coordinates to a metal to form the superstructure subunit (FIG. 10b).

A cyanobacterium—Synechococcus elongatus—was also cultured on one of the artificial siderophore substrate structures (FIG. 9c and FIG. 9d). The photocurrent generation under 1 Sun illumination at light intensity was determined to be 100 W/m². A I-V curve collected for a non-optimized single layer device shows that the photocurrent generation of 92 μA/mm² with the open circuit voltage is 0.15 V (FIG. 9e).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1.-38. (canceled)

39. A method of making a photosynthetic power cell, the method comprising:
a. forming a layer of artificial metallo-siderophores by combining:
i. an amphiphilic siderophore;
ii. a metal ion;
iii. a photosynthetic and/or photoresponsive microorganism, wherein, the combining is performed such that the microorganism is anchored to the amphiphilic siderophore; and
b. adding the layer of artificial metallo-siderophores to the surface of a power cell.

40. The method of claim 39, wherein the amphiphilic siderophore comprises at least one of a catecholate-type artificial siderophore or a hydroxamate-type artificial siderophore.

41. The method of claim 39, wherein the metal ion comprises at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr).

42. The method of claim 39, further comprising combining the amphiphilic siderophore and the metal ion prior to adding the microorganism, wherein the amphiphilic siderophore and the metal ion bind to each other to form a amphiphilic siderophore-metal ion coordinated complex.

43. The method of claim 39, further comprising combining a plurality of amphiphilic siderophore-metal ion coordinated complexes such that the plurality of complexes self-assemble to form a superstructure system.

44. The method of claim 43, wherein the superstructure system is formed in a liquid-liquid interface through a process comprising:
a) directed self-assembly of the amphiphilic siderophore-metal ion coordinated complex; or
b) in-situ coordination programming of the amphiphilic siderophores with the metal ions.

45. The method of claim 39, wherein the photosynthetic and/or photoresponsive microorganism is a bacteria.

46. A composition for biophotonic charge storage comprising:
an amphiphilic siderophore;
a metal ion; and
a photosynthetic and/or photoresponsive microorganism, wherein the microorganism is anchored to the amphiphilic siderophore.

47. The composition of claim 46, wherein the amphiphilic siderophore comprises a catecholate type artificial siderophore or a hydroxamate type artificial siderophore.

48. The composition of claim 46, wherein the metal ion comprises at least one of beryllium (Be), magnesium (Mg), calcium (Ca), Strontium (Sr), Barium (Ba), Radium (Ra), Titanium (Ti), Vanadium (V), Chromium (Cr), Manganese (Mn), Iron (Fe), Cobalt (Co), Nickel (Ni), Copper (Cu), Zinc (Zn), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Thorium (Th), Protactinium (Pa), Uranium (U), Neptunium (Np), Plutonium (Pu), Americium (Am), Curium (Cm), Berkelium (Bk), Californium (Cf), Ensteinium (Es), Fermium (Fm), Mendelevium (Md), Nobelium (No), or Lawrencium (Lr).

49. The composition of claim 46, wherein the amphiphilic siderophore and the metal ion bind to form an amphiphilic siderophore-metal ion coordinated complex.

50. The composition of claim 46, further comprising a superstructure system, the superstructure system comprising the amphiphilic siderophore-metal ion coordinated complex self-assembled with one or more amphiphilic siderophore-metal ion coordinated complexes.

51. The composition of claim 46, wherein the photosynthetic and/or photoresponsive microorganism is a bacteria.

52. A device for biophotonic charge storage comprising:
at least a single layer of a siderophore-metal ion coordinated complex with a photosynthetic and/or photoresponsive microorganism, wherein the single siderophore layer is placed between an anode and a cathode.

53. The device of claim 52, wherein the photosynthetic and/or photoresponsive microorganism is a bacteria.

54. The device of claim 53, wherein the bacteria is a marine purple non-sulfur bacteria.

55. The device of claim 54, wherein the marine purple non-sulfur bacteria is Rhodobacter sphaeroides.

56. The device of claim 53, wherein the bacteria is a cyanobacteria.

57. The device of claim 56, wherein the cyanobacteria is of the Synechocystis genus.

58. The device of claim 52, wherein the initial photovoltage achieved is at least 1.0 V and a capacitance of at least 0.5 F/m².

59. The device of claim 52, wherein the anode is selected from the group consisting of indium tin oxide, indium molybdenum oxide, aluminum-doped zinc oxide, indium-doped cadmium oxide, gallium-doped zinc oxide, and indium-doped zinc oxide.

60. The device of claim 52, wherein the anode is tungsten oxide and molybdenum oxide.

61. The device of claim 52, wherein the cathode is n-Si, aluminum, or gold.
